(19) United States
(12) Patent Application Publication
(10) Pub. No.: US 2008/0089867 A1
(43) Pub. Date: Apr. 17, 2008

(54) Method of increasing retention, survival and proliferation of transplanted cells in vivo

(76) Inventors: Brian Fernandes, Roseville, MN (US); Carl A. Schu, Plymouth, MN (US); Trevor C. Huang, Maple Grove, MN (US); Maura G. Donovan, St. Paul, MN (US)

Correspondence Address: FOX ROTHSCHILD, LLP, 997 LENOX DRIVE, LAWRENCEVILLE, NJ, 08648, US

(21) Appl. No.: 11/580,603

(22) Filed: Oct. 13, 2006

(51) Int. Cl. A61K 35/14; C12N 5/08

(52) U.S. Cl. 424/93.7; 435/366; 435/372

(57) ABSTRACT

A method of increasing retention, survival and proliferation of transplanted cells in diseased or damaged tissue types or organ by providing transplanted cells with autologously-derived platelet cells and forming a autologously-derived platelet gel prior or during administration to the tissue type or organ through a delivery device and immobilizing the transplanted cells in the tissue type or organ system.

### FIELD OF THE INVENTION

The present invention relates to a method to increase the retention, the survival, and the proliferation of transplanted cells in diseased or damaged tissue or organ using autologously-derived platelet cells. The transplanted cells are immobilized in diseased or damaged tissue type or organ using autologously-derived platelet cells, which are gelled at the point of administration or prior to administration through a delivery device.

### BACKGROUND OF THE INVENTION

Coronary heart disease is the leading cause of death in the United States. After a myocardial infarction death of cardiomyocytes results in a left ventricle remodeling and subsequent heart failure. Delivery of cells directly into tissue has been used to treat a variety of tissue disorders including damage to areas of the heart, brain, kidney, liver, gastrointestinal tract, and skin. Direct cell delivery also referred to herein as “transplanted cells” as opposed to systemic delivery has been considered to increase the density of cells in the target area and therefore increase cell survival in tissue, as it is believed that cells must form clusters to survive in tissue. Despite the increase in cell numbers to the target tissue, a result of direct cell delivery versus systemic delivery, only a limited number of delivered cells survive post transplantation into infarcted and/or damaged tissue type or organ. Furthermore, leakage of the delivered cells from the site of the target area is exacerbated in tissue of an organ that undergoes expansion and contraction, such as the heart.

It has been estimated that a very small percentage (˜1 to 10%) of transplanted cells survive within myocardial tissue, with most cells succumbing very early after delivery. Several causative factors, including physical strain during injections, inflammation, apoptosis, and ischemia, and lack of cell retention are likely to be involved. It is desirable that a critical mass of cell survival is necessary to bring about adequate regeneration and transformation of diseased or damaged tissue type or organ into viable, functioning tissue.

Several preclinical approaches to promote cell survival have been recently reported. For example, administration of angiogenic growth factors (VEGF, bFGF, HIF-1), either as proteins or by gene transfer to promote increase blood supply through neovascularization have been described. (Yau and Fung (2002) Circulation 104(suppl 1):218-222; Miyagawa and Sawa (2002) Circulation 105:2556-2561; and Susuki and Murtuza (2001) Circulation 104(suppl 1):207-212.) In addition, tissue engineering approaches for cell survival where scaffolds/matrices are used to provide optimal or improved environments for cells support are described. (Kellar et. al., (2001) Circulation, 104:2063-2068, Leor J, et al., (2000) Circulation 102 (suppl III) 56-61; Li et al., (1999) Circulation 100 (suppl II)63-69.)

Angiogenesis by bone marrow derived cell transplantation in myocardial tissue is also described by Ueno et al., U.S. Pat. No. 6,878,371. Heat shock treatment has been used in the enhancement of graft cell survival enhancement in skeletal myoblast transplantation to the heart. (Suzuki et al., (2000) Circulation, 102(suppl III), 56-61. Transmyocardial revascularization (TMR) laser therapy has also been used to create new bloodlines in oxygen-deprived heart muscle to generate channels for promoting neovascularization.

The combination of skeletal myoblasts with fibrin is disclosed by Christman et.al., (2004) Tissue Engineering, 10(3/4),403-409. The benefit of combining bone marrow mononuclear cells with fibrin is disclosed by Ryu,et.al., Biomaterials, 26, 319-326 (2005).

The disadvantages of these studies are that cells transplanted into cardiac tissue for myocardial regeneration are poorly retained and do not survive in sufficient numbers i.e., less than 10%. Given the above, a need exists for improved methods of retention of transplanted cells.

### SUMMARY OF THE INVENTION

The present invention addresses this and other problems associated with the prior art by providing a method for immobilization of transplanted cells in diseased or damaged tissue type or organ, wherein such method increases the number of injected cells that survive post transplantation. One of the advantages of this approach is that the main components are autologously derived, and other components such as matrices, growth factors, genetic-modification, etc, may not be required. Although the present invention describes by way of example only a cardiac application, Applicants' invention may be applied to any tissue type or organ system.

The present invention provides a method for retention of transplanted cells in diseased and/or damaged tissue type or organ by proliferation and survival of transplanted cells. This method involves administering autologously-derived platelet gel (APG) along with the transplanted cells into the diseased and/or damaged tissue type or organ. The autologously-derived platelet is gelled at the point of administration or prior to administration through a delivery device.

Another aspect of the present invention provides for improving the retention of transplanted cells by administering an APG.

In one aspect of the invention the transplanted cell types may include skeletal myoblasts, or bone marrow derived stem cells, injected with an autologously derived mixture of concentrated blood cells. The autologously derived cells provide the necessary growth factors and cytokines for enhanced survival and proliferation of the transplanted cells.

### DETAILED DESCRIPTION OF THE INVENTION

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

### DEFINITIONS

To aid in the understanding of the invention, the following non-limiting definitions are provided:

The term “transplanted cell” shall mean any non-platelet cell that is delivered to the tissue type or organ to enhance tissue generation.

The term “neo-vascularization” shall mean the development of new capillaries from pre-existing blood vessels, as well as de novo blood vessel formation.

The term “angiogenic agent” shall mean any molecule, cell, or physical stimulus which promotes the growth of blood vessels.

The term “infarcted” refers to tissue that is deprived of its blood supply and dies if left un-treated. As used in this invention infarcted is meant to include damaged tissue type or organ.

The term “retention” refers to ability to keep the transplanted cells in the tissue type or organ.

The term “autologous” refers to the source of the tissue, wherein the tissue is being derived or transferred from the same individual's body, such as, for example, an autologous bone marrow transplant.

The term “autologous cells” refers to cells that are obtained from the recipient of the cells.

The term “allograft” refers to a graft of tissue or the cells obtained from a donor of the same species as, but with a different genetic make-up from, the recipient, as a tissue transplant between two humans.

The term “allogenic” refers to the state of being genetically different although belonging to or obtained from the same species.

The term “treating” or “treatment” of a disease refers to executing a protocol, which may include administering one or more therapeutic agents to a subject (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the subject.

The term “additive” shall mean any molecule, cell, intracellular structure, or any combination thereof.

The term “heart disease” refers to acute and/or chronic cardiac dysfunctions. Heart disease is often associated with a decrease in cardiac contractile function and may be associated with an observable decrease in blood flow to the myocardium (e.g., as a result of coronary artery disease). Manifestations of heart disease include myocardial ischemia, which may result in angina, heart attack and/or congestive heart failure.

The term “subject” shall mean any animal belonging to phylum Chordata, including, without limitation, humans.

The “tissue type” shall mean tissue myocardium, liver and kidney and “organ system” is meant to include by way of example only heart, liver and kidney, lungs, spine, musculoskeletal, etc.

Accordingly, one aspect of the present invention is directed to a method for treating a subject suffering from heart disease, comprising delivering to a region of a wall of the subject's heart which includes a myocardial layer a composition comprising at least two cell populations, wherein said cell populations are substantially immobilized in the myocardium to enhance a therapeutic effect.

Various means exist for delivering therapeutic cells to the heart of a subject suffering from heart disease. Accordingly, another aspect of the present invention is directed to a system for delivering therapeutic cells to the heart of a subject, comprising: means for introducing into said region a composition comprising at least two cell populations, wherein said cell populations are substantially immobilized in the myocardium to enhance a therapeutic effect.

Such systems find particular utility in the treatment of heart disease. Accordingly, another aspect of the present invention is directed to a system for delivering therapeutic cells to the heart of a subject suffering from heart disease, comprising: means for introducing into said region a composition comprising means for introducing into said region a composition comprising two cell populations, wherein said cell populations are substantially immobilized in the myocardium to enhance a therapeutic effect.

Means for delivering the compositions of the present invention into intramyocardial channels are also well known in the art and include both direct and catheter-based injection means. For direct injection, a small bolus of selected composition can be loaded into a micro-syringe, e.g., a 100 μL Hamilton syringe, and applied directly from the outside of the heart.

Preferably, however, the methods and systems of the present invention comprise a catheter means for delivery of the compositions of the present invention. For example, a catheter can be introduced from the femoral artery and steered into the left ventricle, which can be confirmed by fluoroscopy. Alternatively, the catheter can be steered into the right ventricle. In yet another embodiment, injection may be delivered via a catheter and injected to the target area through a wall of a blood vessel adjacent to the target area.

For example, if the target area is a left ventricle (LV) of heart, the catheter, such as, for example, a percutaneous transvenous catheter, may be introduced into different areas of the myocardial wall via either the anterior interventricular. vein, the posterior descending vein or posterolateral vein.

The catheter generally includes an elongated catheter body, suitably an insulative outer sheath which may be made of polyurethane, polytetrafluoroethylene, silicone, or any other acceptable biocompatible polymer, and a standard lumen extending therethrough for the length thereof, which communicates through to a delivery element. The delivery element can be e.g., a hollow needle, a coated delivery surface, a perfusion port(s), a delivery lumen(s), etc. The use of a catheter-based delivery system facilitates composition delivery immediately upon percutaneous myocardial revascularization. In particular, the use of a needle delivery element in conjunction with a catheter-based delivery system allows the operator to perform both mechanical percutaneous myocardial revascularization and composition delivery using a single device.

In one non-limiting example, the suitable catheter is Pioneer CX delivery catheter (Medtronic, Inc., Minneapolis, Minn.). In another embodiment, the catheter is a minimally invasive transvenous catheter, such as, for example, TransAccess LT (available from Medtronic, Inc., Minneapolis, Minn.).

The catheter may be guided to the indicated location by being passed down a steerable or guidable catheter having an accommodating lumen, for example, as disclosed in U.S. Pat. No. 5,030,204, or by means of a fixed configuration guide catheter, such as illustrated in U.S. Pat. No. 5,104,393. Alternately, the catheter may be advanced to the desired location within the heart by means of a deflectable stylet, as disclosed in PCT Patent Application WO 93/04724, or by a deflectable guide wire, as disclosed in U.S. Pat. No. 5,060,660. In yet another embodiment, a needle delivery element may be retracted within a sheath at the time of guiding the catheter into the subject's heart.

The methods of introducing the catheter into the blood vessels are known to persons of ordinary skill in the art. In one non-limiting example, the catheter can be introduced into a femoral vein and advanced into the vessel adjacent to the target area. If the vessel adjacent to the target area of the myocardium is the anterior interventricular artery, the catheter may be advanced from the femoral vein through the right ventricle to the coronary sinus and then to the great cardiac vein. The catheter then penetrates the great cardiac vein and reaches the anterior interventricular artery.

The present invention discloses the combination and co-delivery of two or multiple cell populations, where more than two primary cell types may be used. The first cell population is the transplanted cells and comprise autologous, or allogenic cells. The second cell population comprises autologously derived-platelet cells. Suitable autologously derived-platelet cells are derived from peripheral blood. The autologously derived-platelet cells have instant gelling properties when combined with other agents, such as for example thrombin.

Suitable transplanted cells include, but are not limited to, normal or genetically modified mesenchymal stem cells, hematopoietic stem cells, progenitor cells, cardiomyocytes, myoblasts, procardiomyocytes, skeletal fibroblasts, pericytes, adipose tissue derived cells, umbilical cord derived cells and peripheral blood derived cells.

Transplanted cells obtained from a tissue biopsy may be digested with collagenase or trypsin, for example, to dissociate the cells. Transplanted cells may also be obtained from established cell lines or from embryonic cell sources.

In an embodiment transplanted cells include bone marrow cells of the subject. The transplanted cells from an allograft source, such as, for example, relatives of the subject, or from a xenographic source, preferably, from a member of a close species (for example, if the subject is human, the donor may be a primate, such as, for example, gorilla or chimpanzee). In a preferred embodiment, both the donor and the subject are humans.

The second cell population comprises autologously derived-platelet cells. Such autologously derived-platelet cells may be obtained by methods known to those skilled in the art.

Methods of making an autologously derived-platelet gel is given in U.S. Patent Application 20040022864 to Freyman et. al., which is incorporated herein by reference. Here, whole blood is drawn, either pre-operatively or in the operating room, into a standard blood collection bag containing a citrate-phosphate-dextrose anticoagulant. The blood is then centrifuged by using, for example, a variable-speed centrifuge autotransfusion machine or portable machine, to separate the buffy coat suspended in plasma from the red blood cell pack and platelet-poor plasma fraction or platelet free fraction. This is, the platelet concentrate used for platelet Gel. Depending on the initial platelet counts, it is common to achieve platelet counts in excess of over three to five times baseline counts. Other factors that may be considered in the quality of the autologously derived-platelet gel include, for example, platelet viability and percent retained in the procedure. While white cell content increases 125% with selection for lymphocytes and monocytes, the inclusion of platelets and white cells appears to have several beneficial aspects. For example, white cells confer additional healing cytokines while providing antibacterial activity. On activation with thrombin/calcium to form a coagulum, the platelets interdigitate with the forming of a fibrin web, and developing a gel with adhesiveness and strength materially greater than the plasma alone. The presence of thrombin/calcium also causes platelets to immediately release highly active vasoconstrictors, including beta thromboxane, serotonin and PDGF.

In another embodiment the concentrated mixture of blood cells can be derived with an instrument such as the Magellan (Medtronic Inc., Minneapolis, Minn.). The processing of blood through the Magellan results in platelet rich plasma (PRP), which is a solution that has a platelet count and white blood cell count that is 6× and 3× higher, respectively, than normal levels. This concentrated mixture of blood cells is a rich source of over twenty growth factors and cytokines. The PRP can be activated using thrombin to form the gel.

In one embodiment, in an in vitro model, factors released from activated PRP tremendously increase proliferation rates of cell types such as skeletal myoblasts, smooth muscle cells, fibroblasts, mesenchymal stem cells, endothelial cells when compared to controls. Human APG promoted proliferation of human coronary artery smooth muscle cells over the controls (basal media and growth media) when APG was used with a basal media.

In another embodiment, an in vivo study indicated that activated PRP was able to lead to increased vascularization of surrounding tissue. Human APG led to increased vascularization compared to matrigel in nude mouse model for 7 days.

In another embodiment PRP is used as a source of growth factors. Additionally, besides being a source of growth factors and cytokines the PRP could be a source of blood-borne stem cells. Circulating endothelial progenitor cells (EPC) is one such cell type that is normally present in very low numbers in the blood (<0.01%). EPCs can be cultured to increase the numbers required for transplantation. Additionally, EPCs can be increased in the circulation from 5 to 30 fold following treatment with mobilizing factors such as granulocyte-colony stimulating factor (G-CSF) or granulocyte monocyte colony-stimulating factor (GM-CSF). There is a number of pre-clinical studies, which highlight the neovascularization of heart and limb ischemic models following transplantation or injection of expanded EPCs. One clinical trial (TOPCARE-AMI) involving the use of expanded EPCs, injected into the coronary vasculature, report increase in ejection fraction and regional contractile function, besides improvements in coronary blood flow reserve, at 4 months. There is evidence that blood monocytes can enhance collateral artery growth as well. Monocyte/macrophages are repository of a wide-range of pro-angiogenic factors, including growth factors, inflammatory cytokines and metalloproteinases. Additionally, there is evidence that EPCs may be derived from blood-borne monocytes/macrophages. By concentrating the blood cells the Magellan is capable of increasing the yield of circulating stem cells available for neo-vascularization.

In another embodiment fibrinogen is present in the PRP, which can aid in the retention of the transplanted cells in the tissue type or organ. Following delivery of the cell combination, and subsequent activation of the platelets and WBCs, either through the intrinsic and extrinsic clotting pathways or through contact with activating materials present at the catheter tip, the fibrinogen will be converted into fibrin, a gel like material, thereby trapping and retaining the primary cells at the site of injection. Lack of adequate cell retention is also a reason for poor cell survival. Besides forming a gel, fibrin is an extracellular matrix that is useful for cellular proliferation, migration and integration.

One embodiment of the present invention involves mixing of cultured or same-day processed, autologous, or allogenic, primary stem cells with an enriched fraction of autologous derived blood cells (PRP), generated in the operating room. Thus, a person of the ordinary skill in the art will appreciate that the transplanted cells, such as, for example, primary stem cells and PRP can be mixed in the operating room. This mixture of cell types will be then be co-administered via a syringe or catheter to the target site.

In another embodiment the primary cells can be delivered with platelet poor plasma (PPP). The fibrinogen present in PPP, following activation by thrombin, will form a gel composed of fibrin. This will help to immobilize the cells at the target site.

In one embodiment the PRP fraction obtained from a blood volume of 60 ml with a PRP volumes of 3-10 ml. The platelet yields varied from 4.58 to about 13.5, hematocrit yields varied from 4.9 to about 12.34 and white blood cell yield from 2.3 to about 5. The PRP fraction contained from about 14 different growth factors and cytokines. The growth factors include but not limited to PDGF-BB, PDGF-AA, PDGF-AB, VEGF, FGF-B, HGF, KGF, ANG-2, EGF, TGF-b, TPO, MCP-3, TIMP-1 and BDNF.

In yet another embodiment, the primary cells are mixed with purified populations of blood cells derived from the PRP. That is, the PRP is further processed to generate platelet only, or white blood cell only, populations.

In another embodiment, the enriched blood cell population (PRP) can be added into the tissue type or organ without adding the transplanted cells described earlier. Pre-clinical trials, thus far, have shown improved systolic or diastolic benefits regardless of the cell type used (smooth muscle cells, fibroblasts, cardiomyocytes, skeletal myoblasts, all subsets of bone marrow derived cells, adipose tissue stem cells, embryonic cells, fetal cells). It is very likely that the PRP fraction alone may demonstrate similar beneficial effects, either promoting angiogenesis or myogenesis, or both.

In another embodiment, an in vivo study indicates that platelet free plasma (PFP) can give rise to increased vascularization of surrounding tissue. In another embodiment, the PFP fraction can be mixed with cultured or same-day processed, autologous, or allogenic, primary stem cells. Such a mixture of cell types can be then co-administered via a syringe or catheter to the target site.

In another embodiment, an autologous serum solution, very rich in biological factors, including without limitation, growth factors, antibodies, and cytokines, can be generated from the PRP fraction for delivery with or without the transplanted cells. This can be achieved ex-vivo, in the operating room, by passing the PRP through a syringe loaded with glass wool, the purpose of which is to activate the blood cells. The result is a blood clot from which the biological factors are expressed, removed and filtered. This process is quick and can be done in the operating room. The advantage here is that the resulting enriched serum is free of cellular/membrane components. The role envisioned for the enriched serum is that they will provide the needed survival, growth and differentiation factors needed for the survival, proliferation and integration of the transplanted cells over the short and long term.

The cells useful in the present invention may be administered to the tissue type or organ area via any suitable manner known in the art of direct delivery including engraftment, transplantation, or direct injection via a needle or catheter. Examples of specific devices incorporating injection needles include needle injection catheters, hypodermic needles, biopsy needles, ablation catheters, cannulas and any other type of medically useful needle. Examples of non-needle injection direct delivery devices include, but are not limited to, transmural myocardial revascularization (TMR) devices and percutaneous myocardial revascularization (PMR) devices. Further examples of suitable injection devices include ablation devices and needle-free injectors which propel fluid using a spring or pressurized gas, such as carbon dioxide injection devices.

Non-needle injection devices are also contemplated by the present invention.

It will be understood by one of ordinary skill in the art that other injection devices are contemplated and are within the scope of the invention. Specifically, any device competent to penetrate or separate tissue is contemplated, particularly those that create an opening through which a delivered agent may escape or “leak out,” including for example, a lumen in the device with walls that are shaped such that it can penetrate or separate tissue. A non-limiting example of such a device is an Infiltrator balloon catheter.

The delivery device optionally includes a system within it or working with it to separate platelets. The system may include a device adapted to separate platelets from whole or partially processed blood by filtration. The delivery device of the present invention may integrate a filtration system into a handle or long portion (such as a catheter) of the delivery device. The system of these embodiments preferably separates platelets from larger blood components (such as, blood cells) by using a filter (preferably about a 4 micron filter) to allow platelets and plasma to pass through the filter. Plasma is then preferably removed, to concentrate the platelets, by methods known in the art, such as by using another filter (preferably a less than 1 micron filter) or by using an osmotic or diffusive process.

Another method of separating platelets according to these embodiments, is by platelet specific binding. According to this method, beads or surfaces are used that specifically bind platelets as whole or partial blood passes over them. The bound platelets are then released by a releasing agent or degradation of the beads, surface or binding molecule. The platelets are then concentrated by filtration and/or an osmotic or diffusive process. The delivery devices of the present invention preferably include devices that are capable of separating platelets from whole or partially processed blood.

Specific embodiments according to the methods of the present invention will now be described in the following non-limiting examples. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

### EXAMPLES

### Example 1

IN VIVO ANALYSIS OF APG. APG was evaluated in a nude mice model for 7 days resulting in the formation of a thick fibrovascular capsule enriched with capillaries.

Matrigel is the trade name for a gelatinous protein mixture secreted by mouse tumor cells and marketed by BD Biosciences. This mixture resembles the complex extracellular environment found in many tissues and is used by cell biologists as a substrate for cell culture.

A small volume of chilled (4° C.) Matrigel is dispensed onto plastic tissue culture labware. When incubated at 37° C. the Matrigel proteins self-assemble producing a thin film that covers the surface of the labware. Cells cultured on Matrigel demonstrate complex cellular behavior that is otherwise impossible to observe under laboratory conditions. For example, endothelial cells create intricate spiderweb-like networks on Matrigel coated surfaces but not on plastic surfaces. Such networks are highly suggestive of the microvascular capillary systems that suffuse living tissues with blood. Hence, the process by which endothelial cells construct such networks is of great interest to biological researchers and Matrigel allows them to observe this.

The ability of Matrigel to stimulate complex cell behavior is a consequence of its heterogeneous composition. The chief components of Matrigel are structural proteins such as laminin and collagen which present cultured cells with the adhesive peptide sequences that they would encounter in their natural environment. Also present are growth factors that promote differentiation and proliferation of many cell types. Matrigel contains numerous other proteins in small amounts and its exact composition is unknown.

In the present study APG was compared to Matrigel in a nude mice model for 7 days. APG resulted in the formation of a thick fibrovascular capsule enriched with capillaries. In comparison the matrigel generated a very muted response.

### Example 2

IN-VITRO ANALYSIS OF APG. In cell proliferation assay experiment human coronary artery smooth muscle cells (HCASMC) seeded with human APG and without human APG was studied for 5 days. APG gave increased proliferation of HCASMC over a period of 5 days when compared to basal media and growth factor media. The proliferation indices were significantly greater for APG at three different initial seeding densities of 200, 500 and 10,000 cells.

For HCASMC embedded in APG for 7 days in vitro studies showed cell survival when observed by histology.

In cell proliferation assay for human microvascular endothelical cells observed over four days the cell proliferation indices of APG (with basal medium and growth medium) were significantly greater at time intervals of a day, 2 days, 3 days and 4 days when compared to the basal medium, growth medium. Also the cell proliferation indices of PFP were greater at time intervals of a day, 2 days, 3 days and 4 days when compared to the basal medium, growth medium.

For human skeletal myoblasts cell growth increased when cells were seeded with APG as compared to growth media and basal media.

Although the present invention describes by way of example only a cardiac application, Applicants' invention may be applied to any tissue type or organ system.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method of increasing retention, survival and proliferation of transplanted cells in a diseased and damaged tissue type or organ system comprising:
co-administering the transplanted cells with autologously-derived platelet cells to the tissue type or organ system;
causing the autologously-derived platelet cells to form a autologously-derived platelet gel prior to or during administration to the tissue type or organ system; and
retaining the transplanted cells in the tissue type or organ system.

2. The method of claim 1 wherein the tissue type or organ system is selected from the group consisting of myocardium, liver and kidney, lungs, spine, skeletomuscle system.

3. The method of claim 1 wherein the tissue type or organ system is a myocardium.

4. The method of claim 1 wherein the transplanted cells and autologously-derived platelet cells are co-administered by a delivery device selected from the group consisting of a syringe, a catheter, transmural myocardial revascularization (TMR) devices, percutaneous myocardial revascularization (PMR) devices, ablation devices, needle-free injectors, and multi-needle epicardial injection devices.

5. The method of claim 1, wherein the transplanted cells are selected from the group consisting of normal or genetically modified mesenchymal stem cells, hematopoietic stem cells, progenitor cells, cardiomyocytes, myoblasts, procardiomyocytes, skeletal fibroblasts, pericytes, and a combination thereof.

6. The method of claim 1, wherein the autologously-derived platelet cells are selected from the group consisting of platelet rich plasma (PRP), platelet poor plasma (PPP), platelet free plasma (PFP), and a combination thereof.

7. The method of claim 1, further comprising adding thrombin to the transplanted cells.

8. The method of claim 1, wherein the autologously derived platelet cells are prepared in a Magellan concentrator.

9. The method of claim 1, wherein the autologously derived platelet cells comprises a mixture of enriched growth factors.

10. The method of claim 1, wherein the autologously derived platelet cells comprises a mixture of enriched growth factors selected from the group consisting of PDGF-BB, PDGF-AA, PDGF-AB, VEGF, FGF-B, HGF, KGF, ANG-2, EGF, TGF-b, TPO, MCP-3, TIMP-1 and BDNF.

11. The method of claim 1 wherein the transplanted cells and/or the autologously-derived platelet cells are administered to the tissue type or organ area via engraftment, transplantation, or direct injection.

12. The method of claim 1 wherein the transplanted cells and/or the autologously-derived platelet cells are administered to the tissue type or organ area via a needle or catheter.

13. The method of claim 1 wherein the autologously-derived platelet cells are administered to the tissue type or organ area in a delivery device adapted to separate platelets from whole or partially processed blood by filtration.
